(19) United States
(12) Patent Application Publication
(10) Pub. No.: US 2015/0064287 A1
(43) Pub. Date: Mar. 5, 2015

(54) METHOD FOR TREATING ABNORMAL POLYGLUTAMINE-MEDIATED DISEASES

(71) Applicant: NATIONAL TAIWAN NORMAL UNIVERSITY, Taipei City, TW (US)

(72) Inventors: Guey-Jen LEE-CHEN, Taipei City (TW); Chiung Mei CHEN, Taipei City (TW)

(73) Assignee: NATIONAL TAIWAN NORMAL UNIVERSITY

(21) Appl. No.: 14/462,837

(22) Filed: Aug. 19, 2014

(30) Foreign Application Priority Data
Aug. 27, 2013 (TW) 102130549

(51) Int. Cl. A61K 36/744; A61K 31/7048; A61K 31/7028

(52) U.S. Cl. CPC A61K 36/744; A61K 31/7028; A61K 31/7048
USPC 424/725; 514/35; 514/27

(57) ABSTRACT

The present invention relates to a method for treating abnormal polyglutamine-mediated diseases comprising: administering a pharmaceutical composition comprising one or more selected from the group consisting of a Gardenia jasminoides extract, geniposide and crocin.

### CROSS REFERENCE TO RELATED APPLICATION

This application claims the benefits of the Taiwan Patent Application Serial Number 102130549, filed on Aug. 27, 2013, the subject matter of which is incorporated herein by reference.

### BACKGROUND OF THE INVENTION

1. Field of the Invention

The present invention relates to a method for treating abnormal polyglutamine-mediated diseases comprising: administering a pharmaceutical composition comprising a Gardenia jasminoides extract which can reduce reactive oxygen species to suppress polyglutamine aggregation.

2. Description of Related Art

The spinocerebellar atrophy is referred as spinocerebellar ataxias (SCA), which is a complex group of heterogeneous autosomal dominant neurodegenerative disorder. Spinocerebellar ataxias, caused by expanded CAG repeats encoding a long polyglutamine (polyQ) tract in mutant proteins, and the misfolded polyQ proteins accumulate in the nucleus and cytoplasm of neural cells. The clinical symptom of spinocerebellar atrophy comprises cerebellar degeneration, dysfunctions of nervous system and other parts.

So far, no commercial drug is available for curing or mitigating progressive cerebellar ataxia, which involves following irreversible symptoms: in the beginning, patients cannot properly control their actions; with disease progression, patients gradually become unable to walk and pick up a pen, and eventually unable to speak and swallow; and in the worst case, patients die. However, even the cerebellum, brainstem and spinal cord may atrophy, normal functions of the brain and intelligence are usually not affected, so that the patients can clearly recognize the fact that their bodies gradually become inactive.

Moreover, the modern medical treatment is based on surgery, radiation therapy, chemotherapy, hormonal therapy, biological therapy and so on. It often causes serious side effects on the patients' body, and result in weak patients. On the other hand, many people expect traditional Chinese medical treatment could be helpful since it is relatively mild, generally recognized by people and having a quite high market acceptance.

In view of the gradually increased global population suffering from cerebellar atrophy, what is needed is to find a method capable of reducing the accumulation of polyglutamine used for the manufacturing of pharmaceutical compositions for abnormal accumulated polyglutamine-mediated diseases, serving as an adjuvant therapy for neurodegenerative disease, such as cerebellar atrophy, thereby effectively slowing down the disease progression, and providing the patients with a better quality of life.

### SUMMARY OF THE INVENTION

An object of the present invention is to provide a method for treating polyglutamine-mediated diseases, to thereby serve as an adjuvant therapy for neurodegenerative disease, such as cerebellar atrophy.

Another object of the present invention is to provide a method for reducing reactive oxygen species induced by the accumulation of polyglutamine.

To achieve the above objects, the present invention provides a method for treating abnormal polyglutamine-mediated diseases, comprising: administering a pharmaceutical composition comprising one or more selected from the group consisting of a Gardenia jasminoides extract, geniposide and crocin, wherein geniposide and crocin are the active ingredients extracted from Gardenia jasminoides, but may also be extracted from other Chinese medicine, and the Gardenia jasminoides extract, geniposide and crocin are all commercially available.

In addition, the pharmaceutical composition has the function of reducing reactive oxygen species (ROS). In the polyglutamine-mediated diseases, the misfolded polyglutamine causes increased reactive oxygen species and accumulation of abnormal proteins. Accordingly, the pharmaceutical composition reduces the accumulation of polyglutamine by reducing reactive oxygen species through increasing NFE2L2 (nuclear factor, erythroid 2-like 2) protein expression, and in turn, increasing the expression of the downstream proteins, such as NQO01 (NAD(P)H dehydrogenase, quinone 1), GCLC (glutamate-cysteine ligase, catalytic subunit) and GSTP1 (glutathione S-transferase pi 1).

Examples of the abnormal polyglutamine-mediated diseases are not limited, known to include Huntington's disease, spinal bulbar muscular atrophy and preferably cerebellar atrophy. Eight kinds of cerebellar atrophy are known to be caused by expanded CAG repeats of polyglutamine, including SCA1, SCA2, SCA3, SCA6, SCAT, SCA8, SCA17 and DRPLA (dentatorubral-pallidoluysian atrophy). Such SCAs associated with expanded polyglutamine show a selective and progressive degeneration of cerebellum, brainstem and spinal cord. In the degenerated nerve, expanded polyglutamine proteins significantly accumulate in the nucleus and cytoplasm, resulting in functional insufficiency and degradation of some specific neurons.

The concentration of the Gardenia jasminoides extract is not particularly limited, and can be adjusted depending on the use requirement (for example, disease severity or co-medication). In an embodiment of the present invention, the Gardenia jasminoides extract preferably has a concentration of 1 μg/mL to 100 μg/mL, more preferably 1 μg/mL to 50 μg/mL, and most preferably 1 μg/mL to 10 μg/mL, wherein each of the geniposide and crocin has a concentration of 50 nM to 5 μM, and more preferably 100 ng/mL to 1 μg/mL. In an embodiment of the present invention, geniposide preferably has a concentration of about 500 nM, and crocin preferably has a concentration of about 100 nM.

In the Gardenia jasminoides extract, the active ingredient, geniposide, is transformed into genipin by a reaction of intestinal bacteria (Eubacterium sp. A-44, Reference: Akao, T., Kobashi, K., Aburada, M., 1994. Enzymic studies on the animal and intestinal bacterial metabolism of geniposide. Biol. Pharm. Bull. 17, 1573-1576.), wherein the intestinal bacterial species may be known for those having ordinary skill in the art. Accordingly, the actions and transformation in vivo of the Gardenia jasminoides extract, and the active ingredients, geniposide and crocin, are not limited in the present invention. However, from the following examples, it can be learned that geniposide can reduce the accumulation of polyglutamine and the level of the reactive oxygen species.

In other words, the effective dosage of Gardenia jasminoides extract, geniposide and crocin included in the pharmaceutical composition may be varied according to the administration path, excipients and possibly, the co-used agents, and those having ordinary skill in the art may adjust the dosage thereof to generate the desired effect on the target.

Accordingly, the invention further provides a method for reducing reactive oxygen species, comprising: providing a target; administering a pharmaceutical composition to the target, wherein the pharmaceutical composition comprises one or more selected from the group consisting of a Gardenia jasminoides extract, geniposide and crocin.

The term “reduce” or “reduction” refer to the case that the pharmaceutical composition of the present invention is applied to a subject suffering from polyglutamine-mediated diseases, having symptoms of disease, or having a tendency of development of disease, in order to achieve the treatment, mitigation, slowing, therapy, improvement, or recovery of the tendency of the disease and symptoms.

To implement the method according to the present invention, the above pharmaceutical composition can be administered via oral administering, parenteral administering, inhalation spray administering, topical administering, rectal administering, nasal administering, sublingual administering, vaginal administering, or implanted reservoir, and so on. The term “parenteral” used here refers to subcutaneous injection, intradermal injection, intravenous injection, intramuscular injection, intra-articular injection, intraarterial injection, joint fluid injection, intrathoracic injection, intrathecal injection, injection at morbid site, and intracranial injection or injection technique.

The Gardenia jasminoides extract of the present invention may be purchased from the market, or obtained by water extraction under heating and filtering off residue, but the present invention is not limited thereto. For example, 100 g of dried Gardenia jasminoides and 1500 mL of water are mixed at a temperature of 100° C. for 30 minutes, and sieved using a 100-mesh sieve. Then, the extract is concentrated to 100 mL and filtered through again with a 200-mesh sieve. After that, the extract is dried by a vacuum speed vacuum concentrator and reserved at a temperature of −20° C. Therefore, the pharmaceutical composition formed of the Gardenia jasminoides extract may be processed into a dried extract by using any conventional technique, such as spray drying, freeze-drying, scientific Chinese herbal medicine granulation, and further manufactured into a healthy food and a clinical therapeutic pharmaceutical for the treatment and the prevention of spinocerebellar ataxia. For example, according to the requirement for use, the pharmaceutical composition of the present invention may comprise: at least one of a pharmaceutically acceptable carrier, a diluent, or an excipient in the art. For example, it is encapsulated into liposome to facilitate delivery and absorption. Alternatively, it is diluted with aqueous suspension, dispersion or solution to facilitate injection. Or, it is prepared in a form of a capsule or tablet for storage and carrying. In detail, the composition of the present invention may be formulated in a solid or liquid form. The solid formulation form may include, but is not limited to, powders, granules, tablets, capsules and suppositories. The solid formulation may comprise, but is not limited to, excipients, flavoring agents, binders, preservatives, disintegrants, glidants and fillers. The liquid formation form may include, but is not limited to, water, solutions such as propylene glycol solution, suspensions and emulsions, which may be prepared by mixing with suitable coloring agents, flavoring agents, stabilizers and viscosity-increasing agent.

For example, a powder formulation may be prepared by simply mixing the compound of the present invention with suitable pharmaceutically acceptable excipients such as sucrose, starch and microcrystalline cellulose. A granule formulation may be prepared by mixing the compound of the present invention, suitable pharmaceutically acceptable excipients, suitable pharmaceutically acceptable binders such as polyvinyl pyrrolidone and hydroxypropyl cellulose, followed by wet granulation method using a solvent such as water, ethanol and isopropanol, or dry granulation method using compression force. Also, a tablet formulation may be prepared by mixing the granule formulation with suitable pharmaceutically acceptable glidants such as magnesium stearate, followed by tableting using a tablet machine.

### BRIEF DESCRIPTION OF THE DRAWINGS

The above and other objects, features and other advantages of the present invention will be more clearly understood from the following detailed description taken in conjunction with the accompanying drawings, in which:

FIG. 1A shows a high-performance liquid chromatography (HPLC) pattern at 240 nm of the Gardenia jasminoides extract according to a preferable example of the present invention.

FIG. 1B shows a high-performance liquid chromatography (HPLC) pattern at 440 nm of the Gardenia jasminoides extract according to a preferable example of the present invention.

FIG. 2A shows the cytotoxicity of genipin, geniposide, crocin, the Gardenia jasminoides extract and histone deacetylase inhibitor suberoylanilide hydroxamic acid (SAHA) against HEK-293 cells according to a preferable example of the present invention.

FIG. 2B shows the cytotoxicity of genipin, geniposide, crocin, the Gardenia jasminoides extract and histone deacetylase inhibitor suberoylanilide hydroxamic acid (SAHA) against SH-SY5Y cells according to a preferable example of the present invention.

FIG. 3A shows a Western blot analysis of ATXN3/Q₁₄₋₇₅-GFP protein expression induced by doxycycline according to a preferable example of the present invention.

FIG. 3B shows a Western blot image of NFE2L2 protein expression in ATXN3/Q₁₄₋₇₅-GFP cells according to a preferable example of the present invention.

FIG. 3C shows the quantitative analysis of NFE2L2 protein of FIG. 3B.

FIG. 4 shows the aggregation analysis of ATXN3/Q₇₅-GFP cells treated with genipin, geniposide and crocin, Gardenia jasminoides extract or SAHA according to a preferable example of the present invention.

FIG. 5 shows the Western blot analysis of NFE2L2, NQO1, GCLC and GSTP1 protein expressions in ATXN3/Q₇₅-GFP 293 cells treated with genipin, geniposide, crocin and Gardenia jasminoides extract according to a preferable example of the present invention.

FIG. 6A shows the induced ATXN3/Q₇₅-GFP fluorescence expression of 293 cells treated with genipin, geniposide, crocin, and Gardenia jasminoides extract according to a preferable example of the present invention.

FIG. 6B shows the reactive oxygen species (ROS) analysis of 293 ATXN3/Q₇₅ cells treated with genipin, geniposide, crocin, and Gardenia jasminoides extract according to a preferable example of the present invention.

FIG. 7 shows the radical scavenging activity of kaempferol, crocin and Gardenia jasminoides extract on DPPH according to a preferable example of the present invention.

FIG. 8 shows the cell aggregation analysis of HEK-293T cells transfected with plasmid encoding ATXN3/Q₇₅ and NFE2L2 according to a preferable example of the present invention.

FIGS. 9A, 9B and 9C show the synaptic outgrowth, the number of synaptic processes and the number of synaptic branches of aggregated or non-aggregated SH-SY5Y ATXN3/Q₇₅ cells after treatment with the retinoic acid for one week according to a preferable example of the present invention, respectively.

FIG. 9D shows the cell aggregation analysis of SH-SY5Y ATXN3/Q₇₅ cells treated with genipin, geniposide, crocin and Gardenia jasminoides extract according to a preferable example of the present invention.

### DETAILED DESCRIPTION OF THE PREFERRED EMBODIMENT

In the following description, numerous specific details are set forth to provide a thorough understanding of embodiments of the present disclosure. However, one having an ordinary skill in the art will recognize that embodiments of the disclosure can be practiced without these specific details. In some instances, well-known structures and processes are not described in detail to avoid unnecessarily obscuring embodiments of the present disclosure.

Gardenia jasminoides Extract Preparation and HPLC Analysis

In the following experiment, the Gardenia jasminoides extract was provided by Sun-Ten Pharmaceutical Company (Taipei, Taiwan) as described (Chang et al., 2013; Aqueous extract of Paeonia lactiflora and paeoniflorin as aggregation reducers targeting chaperone in cell models of spinocerebellar ataxia 3. Evidence-based Complementary and Alternative Medicine 2013:471659). High pressure liquid chromatography (HPLC) was performed using a LaChrom Elite HPLC system (Hitachi), consisting of a photo diode array detector. The chromatographic separation of Gardenia jasminoides extract (50 μL, 1 mg/mL) was carried out on a Hypersil ODS (C18) column (250×4.6 mm, 5 μm), eluted with the mixture of water (A), acetonitrile (B) or methanol (C). The linear gradient elution program for A:B:C (v/v/v) was set as follows: 80:5:15 (0-8 min), 70:0:30 (8-30 min), 15:0:85 (30-50 min), 80:5:15 (50-60 min) with a flow rate of 1 mL/min. Then, absorbance was monitored at 240, 440 nm and the scan range for photo diode array was 190-600 nm, wherein geniposide and crocin (50 μL, 0.01-1 mM) were used as reference compounds for Gardenia jasminoides.

Cell Culture and Cell Proliferation Assay

Human embryonic kidney HEK-293 cells (ATCC No. CRL-1573) were cultivated in Dulbecco's modified Eagle's medium (DMEM) containing 10% fetal bovine serum (FBS). Human neuroblastoma SH-SYSY cells (ATCC No. CRL-2266) were maintained in DMEM F12 supplemented with 10% FBS. Cells (5×10⁴/well in 48-well dishes) were cultivated at 37° C. incubator containing 5% CO₂ for 20 hr and treated with different concentrations of the Gardenia jasminoides extract (5-30 mg/mL) or pure compound (100 nM-1 mM). After one day, 20 μL MTT (3,[4,5-dimethylthiazol-2-yl]-2,5-diphenyl-tetrazolium bromide, 5 mg/mL in PBS; Sigma-Aldrich) was added to cells and incubated for 2 hr. The absorbance of the purple formazan dye was measured at 570 nm by a Bio-Tek μQuant Universal Microplate Spectrophotometer.

ATXN3 cDNA Constructs and Isogenic Cell Lines

200 ng of polyadenylation (polyadenylated) RNA isolated from the neuroblastoma SK-N-SH cells was subjected to the reverse transcription using SuperScript™ III reverse transcriptase (purchased from Invitrogen). The forward and reverse primers for amplification of ATXN3/Q₁₄ cDNA (+826 to +1152, NM_{—}004993) were SEQ ID NO: 1, 5′-ATTCAGCTAAGTATGCAAGGTAGTTCCA (the lined letters are Met257 codon), and SEQ ID NO: 2, 5′-CATGCCATGGCATGTTTTTTTCCTTCTGTT (the lined letters are NcoI enzyme site). Then, the amplified 3′ polyQ-containing cDNA fragment (which encoded amino acids 257-361) was cloned into pGEM-T Easy (purchased from Promega) and sequenced. ATXN3/Q₁₄ cDNA cDNA fragment was cut using EcoRI and NcoI, and subcloned into pEGFP-N1 (purchased from Clontech). Then, ATXN3/Q₁₄-EGFP-containing DNA fragment was cut using HindIII-NotI and subcloned into pcDNA5/FRT/TO. In addition, ATXN3/Q₇₅ cDNA was prepared by replacing the ATXN3/Q₁₄ BsmBI-BsmFI fragment of 88 by with the ATXN3/Q₇₅ fragment of 271 by from the cDNA clone of a SCA3 patient.

Isogenic 293 and SH-SY5Y Cell Lines

Flp-In™-293 cells derived from human 293 (Invitrogen) were cultured as described above. The isogenic ATXN3/Q₁₄₋₇₅ cell line was employed, which was formed from subjecting the Flp-In™-293 cells to targeting insertion of the pcDNA5/FRT/TO-ATXN3/Q₁₄ and Q₇₅ recombinant plasmids. These cell lines were grown in medium containing 5 μg/mL blasticidin and 100 μg/mL hygromycin (InvivoGen). In addition, the method for culture in establishment of the human SH-SY5Y derived Flp-In host cell line and the formation of the isogenic ATXN3/Q₁₄₋₇₅ cell line was the same.

ATXN3/Q₇₅ Aggregation Assay

293 ATXN3/Q₇₅-GFP cells were plated into 96-well (2×10⁴/well) dishes, grown for 24 hr and treated with different concentrations of the Gardenia jasminoides extract (1-100 μg/mL) or suberoylanilide hydroxamic acid (SAHA, Cayman Chemical), genipin, crocin (Sigma), geniposide (ChromaDex) (50 nM-5 μM) for 8 hr. Then doxycycline (10 μg/mL, BD) and oxaliplatin (5 μM, Sigma) were added for 6 days. After that, cells were stained with Hoechst 33342 (0.1 μg/mL, Sigma-Aldrich) and aggregation percentage was assessed by high-content analysis (HCA) system (ImageXpressMICRO, Molecular Devices).

SH-SYSY ATXN3/Q₇₅-GFP cells were seeded in 6-well (2×10⁵/well) plate, with all trans retinoic acid (10 μM, Sigma) added at seeding time. At day 2, cells were treated with genipin, geniposide (500 nM), crocin (100 nM) or the Gardenia jasminoides extract (10 μg/mL) for 8 hr, and then doxycycline (5 μg/mL) was added to induce ATXN3/Q₇₅-GFP expression. The cells were kept in the medium containing 10 μM trans retinoic acid, doxycycline and genipin/geniposide/crocin/Gardenia jasminoides extract for one week. After that, cells were stained with Hoechst 33342 (0.1 μg/mL) and aggregation percentage was assessed as described.

Western Blot Analysis

Total proteins were prepared using lysis buffer containing 50 mM Tris-HCl pH8.0, 150 mM NaCl, 1 mM EDTA pH8.0, 1 mM EGTA pH8.0, 0.1% SDS, 0.5% sodium deoxychalate, 1% Triton X-100 and protease inhibitor cocktail (Sigma). Proteins (25 μg) were separated on 10% SDS-polyacrylamide gel electrophoresis and transferred onto nitrocellulose membranes by reverse electrophoresis. After blocking, the membrane was probed with NFE2L2 (1:500 dilution, Santa Cruz), NQO1 (NAD(P)H dehydrogenase, quinone 1; 1:1000 dilution, Sigma), GCLC (glutamate-cysteine ligase, catalytic subunit; 1:100 dilution, Abcam), GSTP1 (glutathione S-transferase pi 1; 1:1000 dilution, Abcam) or β-actin (ACTB) (1:5000 dilution, Millipore) at 4° C. overnight. Then the immune complexes were detected by horseradish peroxidase-conjugated goat anti-mouse or goat anti-rabbit IgG antibody (1:5000 dilution, GeneTex) and chemiluminescent substrate (Millipore).

ROS Analysis

293 ATXN3/Q₇₅-GFP cells were plated into 6-well (10⁵/well) dishes, grown for 24 hr and treated with different concentrations of the Gardenia jasminoides extract (10 μg/mL) or genipin, geniposide (500 nM), crocin (100 nM) for 8 hr. Then doxycycline (10 μg/mL) and oxaliplatin (2.5 μM) were added to the medium for ATXN3/Q₇₅-GFP expression and aggregate accumulation. Six days later, fluorogenic CellROX™ Deep Red Reagent (5 μM, Molecular Probes) designed to reliably measure ROS in live cells was added to the cells and incubated at 37° C. for 30 min. The cells were then washed with PBS and analyzed for green (GFP) and red (ROS) fluorescence on a flow cytometry (Becton-Dickinson), with excitation/emission wavelengths at 488/507 (green) and 640/665 nm (red). 5×10⁴ cells in each sample were analyzed.

1,1-diphenyl-2-picryl hydrazyl (DPPH) Assay

100 μM DPPH radical solution (purchased from Sigma) and 10-200 μM of genipin, geniposide, crocin or 0.2-4 mg/mL of Gardenia jasminoides extract were vortexed for 15 seconds and then left to stand at room temperature for 30 min. Then, the scavenging capacity was measured by monitoring the decrease in absorbance at 517 nm by a Thermo Scientific Multiskan GO Microplate Spectrophotometer. The radical scavenging activity was calculated using the formula: 1-(absorbance of sample/absorbance of control)×100%. The antioxidative activity was expressed as EC₅₀, which was defined as the concentration of the compounds required for inhibition of the formation of DPPH radicals by 50%.

The NFE2L2 cDNA (BC011558) in pOTB7 was obtained from Bioresource Collection and Research Center (BCRC), Food Industry Research and Development Institute, Taiwan. The cDNA was excised with BamHI and XhoI and subcloned into pcDNA3 (Invitrogen). For transient overexpression, cells were plated into 12-well (5×10⁵/well) dishes, grown for 20 hr, and transfected with using T-Pro reagent with pcDNAS/FRT/TO-ATXN3/Q₇₅ and overnight later with pcDNA3-NFE2L2 or pcDNA3 vector plasmids (1.5 μg each).

Statistical Analysis

For each set of values, data were expressed as the means±standard deviation (SD). Three independent experiments were performed and non-categorical variables were compared using the Student's t-test. All P-values were two-tailed, with values of P<0.05 considered significant.

Results

### Gardenia jasminoides Extract, Constituents and IC₅₀ Cytotoxicity

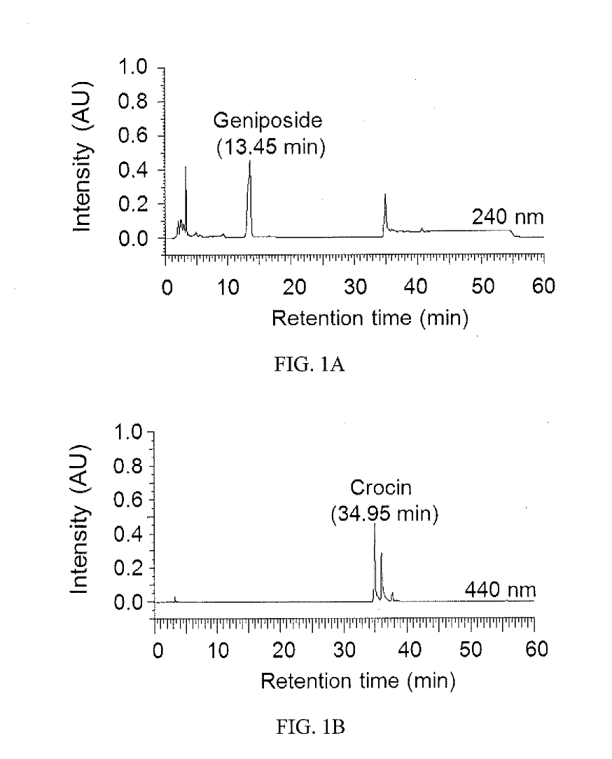

The chemical properties of the Gardenia jasminoides extract were analyzed and quantified by full-spectrum analytic HPLC. The chromatographic patterns in FIG. 1A show peaks at 240 nm corresponding to the retention time compatible with geniposide. The chromatographic patterns in FIG. 1B show peaks at 440 nm corresponding to the retention time compatible with crocin. The amounts of geniposide and crocin in 1 g/mL of Gardenia jasminoides extract were 2.33% and 1.03%, respectively.

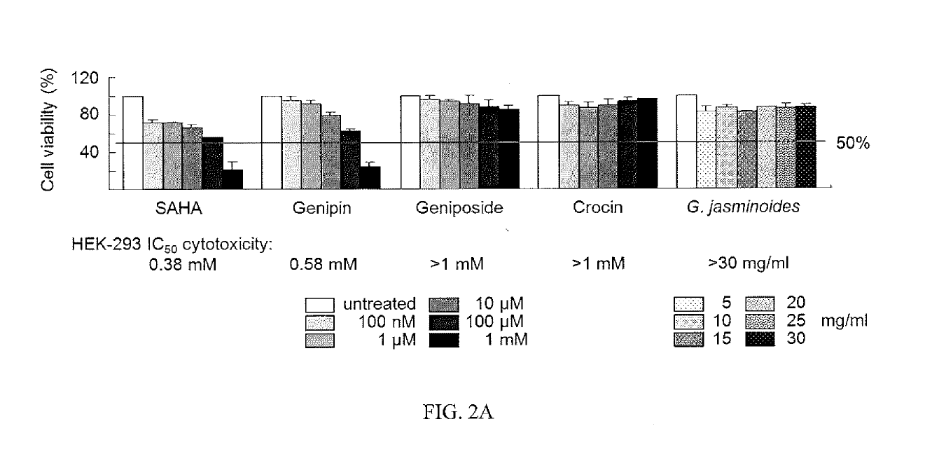

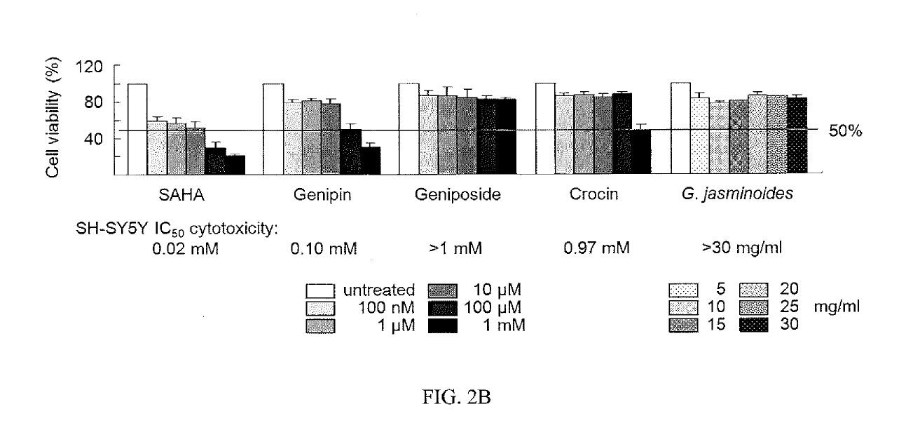

In MTT assays, the cytotoxicity of the Gardenia jasminoides extract, geniposide and crocin against human embryonic kidney 293 and human neuroblastoma SH-SY5Y cells was shown in FIGS. 2A and 2B, wherein SAHA, a histone deacetylase inhibitor known to reduce SDS-insoluble polyQ aggregates, was included for comparison. Accordingly, SAHA and genipin had an IC₅₀ of 0.38 mM and 0.58 mM, respectively in HEK-293 cells and 0.02 mM and 0.10 mM, respectively in SH-SY5Y cells. Both Gardenia jasminoides extract and its constituents geniposide and crocin had an IC₅₀ higher than or close to the highest concentration tested (>30 mg/mL for Gardenia jasminoides, >1 mM for geniposide and >1 mM (HEK-293)/0.97 mM (SH-SY5Y) for crocin, suggesting their very low cytotoxicity.

293 ATXN3/Q₇₅ Cells and Reduced NFE2L2 Expression

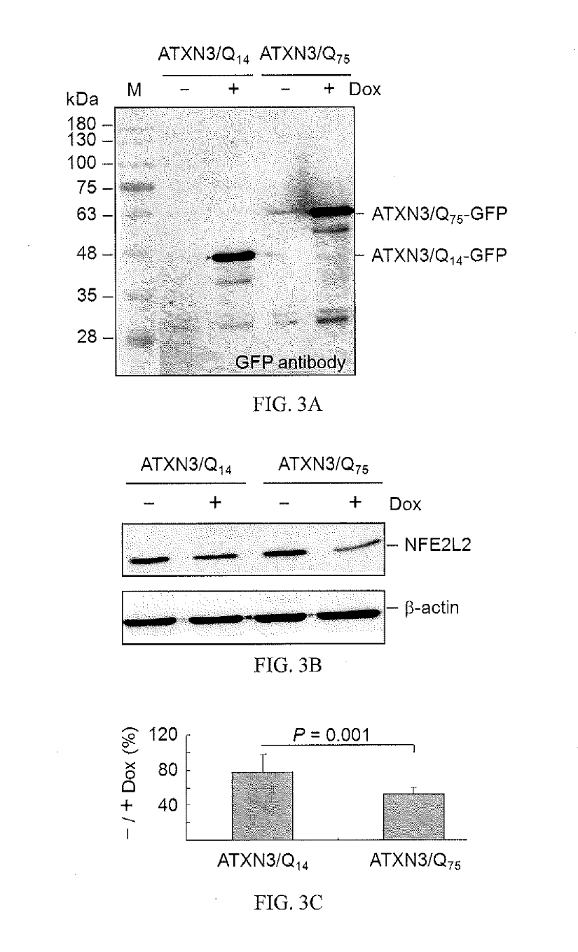

In this experiment, ATXN3/Q₁₄₋₇₅ fragment with C terminal marked by GFP was established, to form the Flp-In 293 cells with ATXN3/Q₁₄₋₇₅-GFP expression in an inducible fashion, wherein ATXN3/Q₁₄ was the control group of normal protein expression. As shown in FIG. 3A, in the doxycycline-induced ATXN3 cells, GFP antibody detected 40 kDa of ATXN3/Q₁₄-GFP and 57 kDa of the ATXN3/Q₇₅-GFP proteins. According to the images under fluorescence microscope, ATXN3/Q₁₄-GFP expression mainly exhibited diffuse distribution, while ATXN3/Q₇₅-GFP expression mainly exhibited aggregation (not shown).

PolyQ mutation may induce reactive oxygen species and NFE2L2 plays a central role in the regulation of the cellular redox status. Please refer to FIGS. 3B and 3C, showing the Western blot image and quantitative analysis of NFE2L2 protein expressed by ATXN3/Q₁₄₋₇₅-GFP cells induced by doxycycline. NFE2L2 expression in the ATXN3/Q₇₅-GFP cells was significantly reduced as compared to that of the ATXN3/Q₁₄-GFP cells (78% vs. 52%, P=0.001).

Effects of Gardenia jasminoides Extract and its Derivatives on 293 ATXN3/Q₇₅ Aggregation

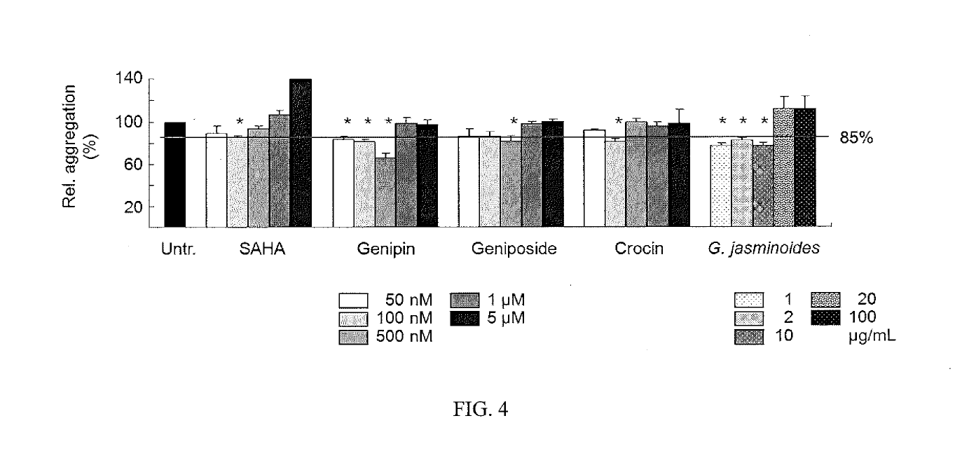

In this experiment, influence of genipin, geniposide, crocin, Gardenia jasminoides extract, and SAHA on ATXN3/Q₇₅ was tested using ATXN3/Q₇₅-GFP cells. As shown in FIG. 4, as a positive control, HDAC inhibitor SAHA reduced the ATXN3/Q₇₅ aggregation to 85.9% (at 100 nM, P=0.002) as compared to untreated cells. Additional to good aggregation-inhibitory potential seen with genipin (83.64-66.1% at 50 nM-500 nM, P=0.004-0.009), Gardenia jasminoides (82.8-77.3% at 1-10 μg/mL, P=0.004-0.008), geniposide (82.3% at 500 nM, P=0.028) and crocin (81.5% at 100 nM, P=0.007) also had a greater aggregation reduction potential than SAHA. The IC₅₀ cytotoxicity/effective (reduced the ATXN3/Q₇₅ aggregation to 86% or lower) dose ratio of SAHA, genipin, geniposide, crocin and Gardenia jasminoides extract are 3800, 11600, >2000, >10000 and >30000, respectively. Since 10 μg/ml of Gardenia jasminoides extract contained 600 nM geniposide and 106 nM crocin, the results of experiments shown aggregation reduction potential of 500 nM for geniposide and 100 nM for crocin, and both geniposide and crocin can be regarded as major active components for the aggregation inhibition in Gardenia jasminoides.

Effects of Gardenia jasminoides Extract and its Derivatives on NFE2L2, NQO1, GCLC and GSTP1 Expression

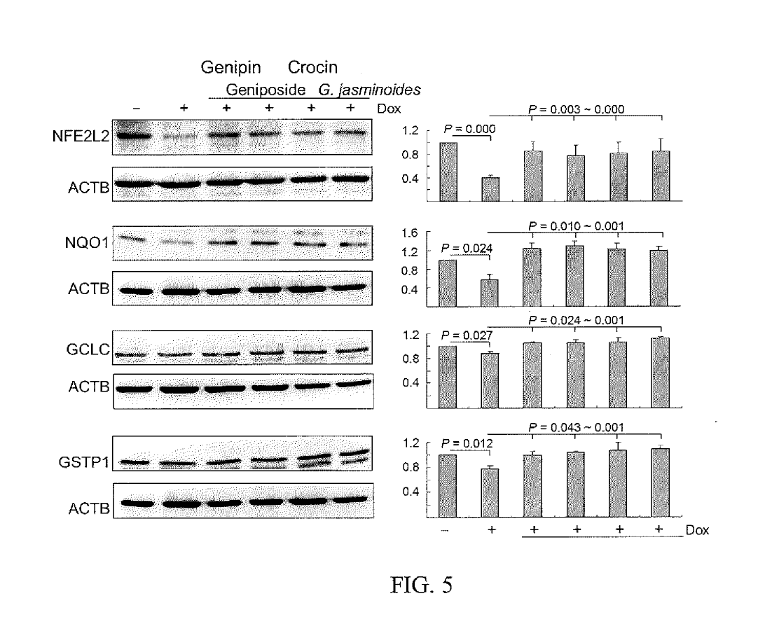

The protein expression levels of NFE2L2, NQO1, GCLC and GSTP1 were compared under addition of the Gardenia jasminoides extract and its active ingredients and/or doxycycline, to examine if the Gardenia jasminoides extract and its active ingredients up-regulated the NFE2L2 and the downstream protein expression levels of NQO1, GCLC and GSTP1. ATXN3/Q₇₅ cells were treated with genipin, geniposide, crocin and G. jasminoides extract for 8 hr and induced by doxycycline for 6 days. FIG. 5 shows the Western blot analysis of NFE2L2, NQO1, GCLC and GSTP1 protein expressions, wherein the ACTB group was control. The results indicated that: after 6 days of induction by doxycycline, the protein expression of NFE2L2 (40%, P=0.000), NQO1 (58%, P=0.024), GCLC (88%, P=0.027) and GSTP1 (78%, P=0.012) were significantly attenuated. This reduction can be rescued by the addition of genipin (500 nM), geniposide (500 nM), crocin (100 nM) or Gardenia jasminoides (10 μg/mL), with significantly increased NFE2L2 (78%-86%, P=0.003-0.000), NQO1 (137%-154%, P=0.002-0.001), GCLC (105%-113%, P=0.024-0.001) and GSTP1 (99%-109%, P=0.043-0.001) protein expression. These findings indicated that genipin, geniposide, crocin and Gardenia jasminoides up-regulated NFE2L2 and downstream NQO1, GCLC, and GSTP1 expression to reduce ATXN3/Q₇₅ aggregation.

Effects of Gardenia jasminoides Extract and its Derivatives on ROS Production

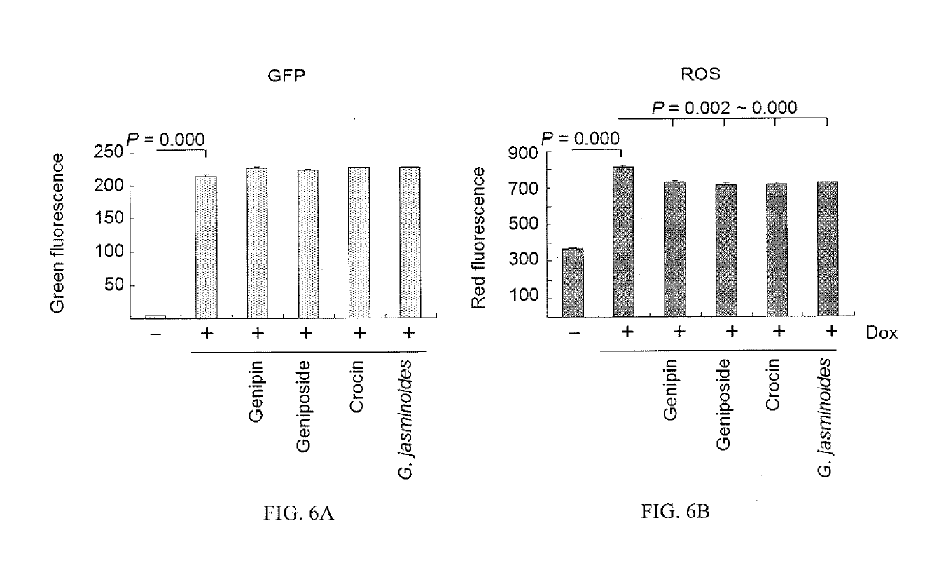

The fluorescence and ROS levels of ATXN3/Q₇₅-GFP cells were compared under the addition of the Gardenia jasminoides extract and its active ingredients and/or doxycycline. As shown in FIGS. 6A and 6B, after 6 days with or without pretreatment of genipin, geniposide, crocin and G jasminoides extract and the use of doxycycline, the ATXN3/Q₇₅-GFP fluorescence expression between each groups had no significant difference (215-228, P=0.169-0.274), but the pretreatment of Gardenia jasminoides extract and its active ingredients significantly ameliorated oxidative stress induced by ATXN3/Q₇₅ (red fluorescence from 814 to 715-736, P=0.002-0.000).

Radical Scavenging Activity of Gardenia jasminoides Extract and its Derivatives

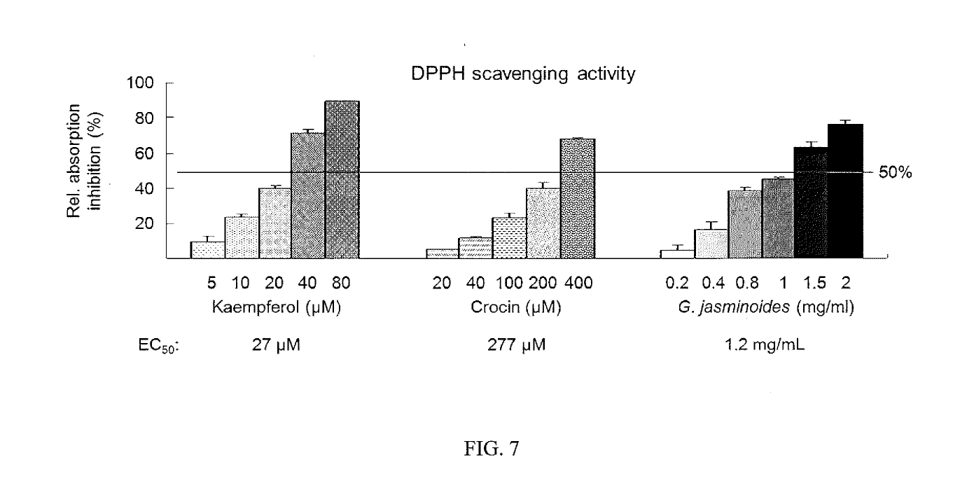

In this experiment, kaempferol with strong antioxidant property was chosen as the reference antioxidant. As shown in FIG. 7, no detectable DPPH scavenging activity was seen with genipin and geniposide (data not shown), and kaempferol, crocin and Gardenia jasminoides extract had an EC₅₀ of 27 μM, 277 μM and 1.2 mg/mL, respectively. Accordingly, since 1.2 mg/ml of Gardenia jasminoides extract contained 12.7 μM crocin and EC₅₀ DPPH scavenging activity of 277 μM for crocin, it mean that crocin seemed not a major contributor to the antioxidant activity in the Gardenia jasminoides extract.

NFE2L2 Overexpression to Reduce ATXN3/Q₇₅ Aggregation

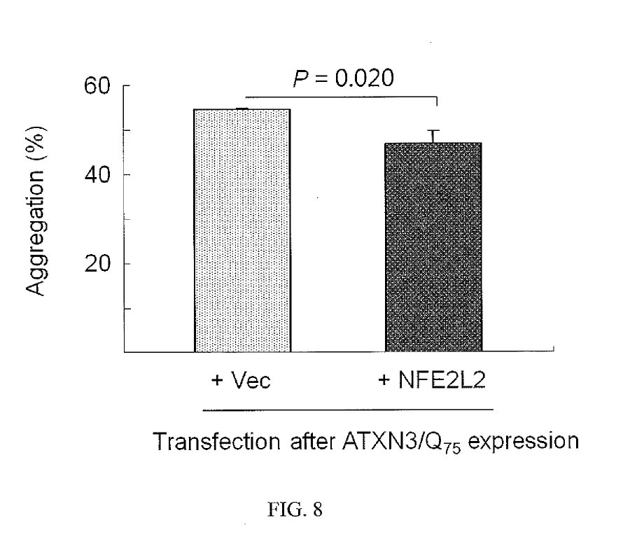

As shown in FIG. 8, NFE2L2 shows significantly decreased aggregation of ATXN3/Q₇₅ cells (44.0% vs. 54.5% P=0.020). The result indicated that genipin, geniposide, crocin and G. jasminoides extract inhibited aggregation by up-regulating NFE2L2 and its downstream gene expressions.

Effect of Gardenia jasminoides Extract and its Derivatives on SH-SY5Y ATXN3/Q₇₅ Aggregation

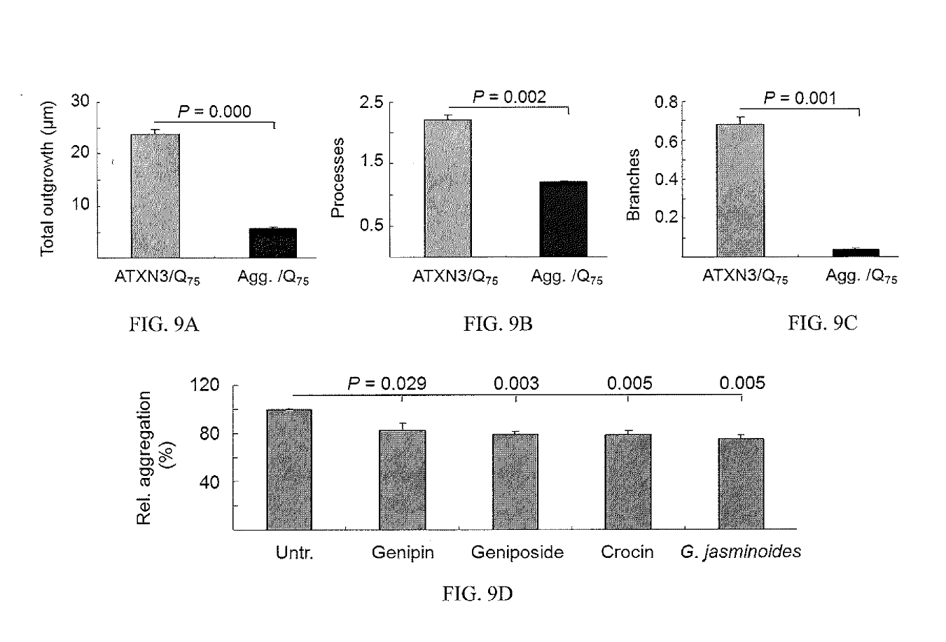

After differentiation of ATXN3/Q₁₄₋₇₅-GFP SH-SYSY cells using retinoic acid for 7 days, it can be found that the induced ATXN3/Q₇₅-GFP formed aggregates in -1% differentiated neurons, whereas no aggregate was seen in ATXN3/Q₁₄-GFP cells, and the aggregates were increased with time (not shown). As shown in FIGS. 9A to 9C, for ATXN3/Q₇₅-GFP expressing cells, significant less total outgrowth, processes and branches in aggregated cells were observed compared to non-aggregated cells, with 5.62 μm vs. 23.88 μm for total outgrowth (P=0.000), 1.21 vs. 2.22 for process (P=0.002), and 0.04 vs. 0.68 for branch (P=0.001). Referring to FIG. 9D, the treatment of genipin, geniposide, crocin and Gardenia jasminoides extract leaded to 18%, 21%, 21%, and 25% of aggregation reduction, respectively in ATXN3/Q₇₅ expressed neuronal cells, accompanying increased NFE2L2 expression (data not shown).

It should be understood that these examples are merely illustrative of the present invention and the scope of the invention should not be construed to be defined thereby, and the scope of the present invention will be limited only by the appended claims.

What is claimed is:

1. A method for treating abnormal polyglutamine-mediated diseases, comprising: administering a pharmaceutical composition comprising one or more selected from the group consisting of a Gardenia jasminoides extract, geniposide and crocin.

2. The method of claim 1, wherein the Gardenia jasminoides extract has a concentration of 1 μg/mL to 100 μg/mL.

3. The method of claim 1, wherein each of the geniposide and crocin has a concentration of 50 nM to 5 μM.

4. The method of claim 1, wherein the pharmaceutical composition further comprises: at least one pharmaceutically acceptable carrier, a diluent, or an excipient.

5. The method of claim 1, wherein Gardenia jasminoides in the pharmaceutical composition is an extract obtained by water extraction under heating and filtering off residue.

6. The method of claim 1, wherein geniposide is transformed into genipin by a reaction of intestinal bacteria.

7. The method of claim 1, wherein the abnormal polyglutamine-mediated disease is spinocerebellar ataxia.

8. A method for reducing reactive oxygen species, comprising:
administering a pharmaceutical composition comprising one or more selected from the group consisting of a Gardenia jasminoides extract, geniposide and crocin.

9. The method of claim 8, wherein the Gardenia jasminoides extract has a concentration of 1 μg/mL to 100 μg/mL.

10. The method of claim 8, wherein each of the geniposide and crocin has a concentration of 50 nM to 5 μM.

11. The method of claim 8, wherein the pharmaceutical composition further comprises: at least one pharmaceutically acceptable carrier, a diluent, or an excipient.

12. The method of claim 8, wherein Gardenia jasminoides in the pharmaceutical composition is an extract obtained by water extraction under heating and filtering off residue.

13. The method of claim 8, wherein geniposide is transformed into genipin by a reaction of intestinal bacteria.

14. The method of claim 8, wherein the abnormal polyglutamine-mediated disease is spinocerebellar ataxia.

15. The method of claim 8, wherein the reactive oxygen species is reduced by the pharmaceutical composition through an increase of NFE2L2 (Nuclear factor, erythroid 2-like 2) protein expression.

16. The method of claim 8, wherein the reactive oxygen species is reduced by the pharmaceutical composition through an increase of NQO1 (NAD(P)H dehydrogenase, quinone 1), GCLC (glutamate-cysteine ligase, catalytic subunit), and GSTP1 (glutathione S-transferase pi 1) proteins expression.


SEQUENCE LISTING (2 sequences)

SEQ ID NO: 1 - DNA, 28 bp, Artificial Sequence
  Synthesized primer
attcagctaagtatgcaaggtagttcca

SEQ ID NO: 2 - DNA, 30 bp, Artificial Sequence
  Synthesized primer
catgccatggcatgtttttttccttctgtt